(19) United States
(12) Patent Application Publication
(10) Pub. No.: US 2022/0331448 A1
(43) Pub. Date: Oct. 20, 2022

(54) COMPOSITIONS AND METHODS FOR TREATING SUCCINIC SEMIALDEHYDE DEHYDROGENASE DEFICIENCY (SSADHD)

(71) Applicant: Washington State University, Pullman, WA (US)

(72) Inventors: Kara Rain VOGEL, Madison, WI (US); Kenneth Michael Gibson, Spokane, WA (US); Garret Robert AINSLIE, Burlingame, CA (US)

(21) Appl. No.: 17/708,799

(22) Filed: Mar. 30, 2022

Related U.S. Application Data
(63) Continuation of application No. 16/969,927, filed on Aug. 13, 2020, now abandoned.
(60) Provisional application No. 62/634,092, filed on Feb. 22, 2018.

(51) Int. Cl. A61K 48/00; A61K 9/00; A61K 31/18; A61K 31/197; A61K 31/436

(52) U.S. Cl. CPC A61K 48/005; A61K 9/0019; A61K 31/18; A61K 31/197; A61K 31/436; A61K 48/0025

(57) ABSTRACT

Provided herein are compositions and methods for treating succinic semialdehyde dehydrogenase deficiency (SSADHD). Compositions may include a gene encoding a functional succinic semialdehyde dehydrogenase (SSADH) enzyme, such as ALDH5A1, operably linked to a targeting vector. The functional SSADII enzyme is envisioned to lower the levels of circulating gamma-hydroxybutyric acid (GHB) and γ-aminobutyric acid (GABA). In some embodiments, combination therapies are envisioned, comprising administering to the subject therapeutically effective amounts of a combination of a composition comprising a gene encoding a functional SSADII enzyme operably linked to a targeting vector; one or more mTOR inhibitors; and a GABA-T inhibitor. Suitable mTOR inhibitors include rapamycin, while suitable GABA-T inhibitors include vigabatrin.

### CROSS-REFERENCE TO RELATED APPLICATIONS

This application claims the benefit of U.S. Provisional Application No. 62/634,092, filed Feb. 22, 2018. The entire contents of the above-identified application are hereby fully incorporated herein by reference.

### STATEMENT REGARDING FEDERALLY SPONSORED RESEARCH

This invention was made with government support under Grant No.(s) NS082286, NS098856, NS085369, and EY027476 awarded by the National Institutes of Health. The government has certain rights in the invention.

### TECHNICAL FIELD

The subject matter disclosed herein is generally directed to compositions and methods for treating neurological disorders.

### BACKGROUND

Progress in understanding the pathophysiology and clinical features of succinic semialdehyde dehydrogenase deficiency (SSADHD) has continued since its description in the 1980s (Jakobs et al. Clin Chim Acta 111:169-178 (1981); Gibson et al. Clin Chim Acta 133:33-42 (1983)). A major advance came in 1999 with the development of a murine knockout model, so-called aldehyde dehydrogenase 5al (aldh5a1−/−) mice (Hogema et al. 2000). While the phenotype of this animal model is severe, with early lethality at ˜3 weeks of life, it has provided insights into GABAergic, glutamatergic, GHBergic, metabolic, oxidative stress, and other parameters otherwise unavailable (Gupta et al. J Pharmacol Exp Ther 302:180-187 (2002); Cortez et al. Pharmacol Biochem Behav 79:547-553 (2004); Buzzi et al. Brain Res 1090:15-22 (2006); Wu et al. Ann Neurol 59:42-52 (2006); Jansen et al. BMC Dev Biol 8:112 (2008); Pearl et al. J Inherit Metab Dis 32:343-352 (2009); Vogel et al. Ann Clin Transl Neurol 2:699-706 (2015); Vogel et al. J Inherit Metab Dis 39:877-886 (2016); Vogel et al. Clin Pharmacol Ther 101:458-461 (2017); Vogel et al. Toxicol In Vitro 40:196-202 (2017); Vogel et al. Pediatr Neurol 66:44-52.e1. (2017); Vogel et al. Biochim Biophys Acta 1863:33-42 (2017); Vogel et al. Toxicol In Vitro 46:203-212 (2017); Vogel et al. PLoS One 12(10):e0186919 (2017)). Thus far, therapeutics that rescue this model from premature lethality include GABAB and GHB receptor antagonists, the non-physiological amino acid taurine, the antiepileptic agent vigabatrin, the ketogenic diet, and rapalog agents such as Torin 2, the latter an mTOR inhibitor. SGS742, a GABABR antagonist, is the subject of an ongoing clinical trial (www.clinicaltrials.gov; NCT02019667).

GABA, the major central inhibitory neurotransmitter (Schousboe and Waagepetersen Prog Brain Res 160:9-19 (2007)) and its related structural analog, gamma-hydroxybutyric acid (GHB), accumulate to supraphysiological levels in SSADHD (Malaspina et al. Neurochem Int 99:72-84 (2016)) (FIG. 1). The degree to which each contribute to pathophysiology remains unknown. However, emerging new roles for GABA exist beyond that of inhibitory neurotransmitter, including neuro-endocrine effects along the gut-brain axis, autophagy, circadian rhythms, and others (Kilb Neuroscientist 18:613-630 (2012); Lakhani et al. EMBO Mol Med 6:551-566 (2014); Chellappa et al. Sci Rep 6:33661 (2016); Mittal et al. J Cell Physiol 232:2359-2372 (2017)). These roles provide novel opportunities to explore pathomechanisms in SSADHD. Novel directions for research and preclinical drug development for treatment of SSADHD are needed.

### SUMMARY

In one aspect, provided herein is a composition for treating succinic semialdehyde dehydrogenase deficiency (SSADHD), comprising a gene encoding a functional succinic semialdehyde dehydrogenase (SSADH) enzyme operably linked to a targeting vector. In some embodiments, the gene may be ALDH5A1.

In some embodiments, the targeting vector is a viral vector. Suitable viral vectors include, but are not necessarily limited to, retroviral vectors, adenoviral vectors, or adeno-associated viral vectors. In specific embodiments, the retroviral vector is a lentiviral vector.

In some embodiments, the targeting vector targets the liver.

In some embodiments, the functional SSADH enzyme lowers the levels of circulating gamma-hydroxybutyric acid (GHB) and γ-aminobutyric acid (GABA). In some embodiments, the composition does not cross the blood brain barrier.

In another aspect, the invention provides a method of treating SSADHD in a subject in need thereof, comprising administering a therapeutically effective amount of any of the compositions described herein. In some embodiments, the therapeutically effective amount may comprise a range of 1-10,000 μg functional SSADH enzyme per kg of body weight per day. In some embodiments, the composition is administered once per week, bi-weekly, or once a month. In some embodiments, the composition is administered intravenously.

In yet another aspect, the invention provides a method of treating SSADHD in a subject in need thereof, comprising administering to the subject therapeutically effective amounts of: a composition comprising a gene encoding a functional SSADH enzyme operably linked to a targeting vector; one or more mTOR inhibitors; a GABA-T inhibitor; or a combination thereof.

Suitable mTOR inhibitors may include, but are not necessarily limited to, rapamycin, sirolimus, temsirolimus, everolimus, and ridaforolimus, Torin 1, and Torin 2. In specific embodiments, the mTOR inhibitor is rapamycin. In specific embodiments, the GABA-T inhibitor is vigabatrin.

In some embodiments, combination therapies are envisioned, comprising administering therapeutically effective amounts of: Torin 2, Vigabatrin, and a composition comprising a gene encoding a functional SSADH enzyme operably linked to a targeting vector.

In specific embodiments, the therapeutically effective amount of Torin 2 and/or Vigabatrin comprises 1-25 μg per kg of body weight per day. In some embodiments, the Torin 2 and/or Vigabatrin are administered two or three times a day.

In some embodiments, the subject may have increased levels of circulating metabolites. Such circulating metabolites may include, but are not necessarily limited to, GHB, GABA, or both.

In yet another aspect, the invention provides a method of treating SSADHD in a subject in need thereof, comprising administering a therapeutically effective amount of an NKCC1 inhibitor to the subject. NKCC1 inhibitors may include, but are not necessarily limited to, bumetanide, allopregnanolone, pregnanolone, progesterone, gaboxadol, etifoxine, XBD-173, FG-7142, gabazine, isoniazid, encenicline, and AVL-3288. In specific embodiments, the NKCC1 inhibitor is bumetanide.

These and other aspects, objects, features, and advantages of the example embodiments will become apparent to those having ordinary skill in the art upon consideration of the following detailed description of illustrated example embodiments.

### BRIEF DESCRIPTION OF THE DRAWINGS

An understanding of the features and advantages of the present invention will be obtained by reference to the following detailed description that sets forth illustrative embodiments, in which the principles of the invention may be utilized, and the accompanying drawings of which:

FIG. 1—A schematic illustrating GABA metabolism and intracellular interactions. The site of the defect in patients with SSADHD is indicated by “X”. Abbreviations: GABA, γ-aminobutyric acid; GABAAR, ionotropic GABAA receptors; GABABR, metabotropic GABAB receptors. GABA-T, GABA-transaminase; SSA, succinic semialdehyde; AKR7a2, aldo-keto reductase 7a2; GHB, γ-hydroxybutyric acid; cAMP, cyclic AMP; NKCC1, sodium potassium chloride cotransporter 1; KCC2, neuronal potassium chloride cotransporter 2. In SSADHD, GABA, SSA and GHB accumulate (↑). Increased GABA and GHB activate GABAA and GABAB receptors and a putative GHB receptor (molecular identification unknown). However, a compensatory downregulation of GABA and GHB receptors 4) has been reported in SSADHD suggesting excess GABA does not lead to increased inhibitory neurotransmission in vivo. NKCC1 and KCC2 control transmembrane chloride gradient and determine GABAA receptor directional transmembrane chloride flux. In experimental SSADHD, the expression ratio of NKCC1 and KCC2 (NKCC1/KCC2) is elevated (Vogel et al. Pediatr Neurol 66:44-52.e1. (2017c)), suggesting that activation of GABAA receptors causes chloride efflux from brain cells, membrane depolarization and activation of neurotransmission (see FIG. 3 for further details). NKCC1 inhibitors like bumetanide lower intracellular chloride concentration. In SSADHD, bumetanide may thus restore GABA inhibitory neurotransmission activity and efficiently suppress seizures. Last, in SSADHD as well as in vigabatrin-treated animals, increased GABA activates the mTOR pathway with secondarily increased mitochondria number, oxidative stress, autophagy and mitophagy. mTOR inhibitors such as torin 1 and torin 2 improve GABA-induced, mTOR-pathway mediated intracellular defects and significantly prolong the lifespan of aldh5a1-deficient mice.

FIG. 2—A graph illustrating the cortical gene expression profile of solute carriers (Slc) in aldh5a1−/− mice following NCS-382 administration (7 days, q.i.d., 300 mg/kg). Relative levels are displayed, normalized to control aldh5a1−/− mice receiving vehicle. Functional role of carriers: *17a6, *17a7, *17a8, vesicular glutamate (glu) transporter, glu cotransporter, and glu transporter 3, respectively; 1a1, *1a2, 1a3, 1a4, excitatory amino acid transporters 3, 2, 1 and 4, respectively; *32a1, GABA vesicular transporter; 38a1, Na+-coupled amino acid transporter 1 (glutamine transport); *6a1, plasma membrane GABA transporter; *6a11, 6a12, 6a13, Na+-dependent GABA presynaptic terminal reuptake, Na+/Cl−-dependent betaine, and Na+/Cl−-dependent GABA 2 transporters, respectively; 7a11, glutamate-cysteine antiporter. Asterisked genes demonstrated correction of expression following chronic NCS-382 administration. Values represent pooled data of biological triplicates (n=3 animals each, NCS-382 and vehicle).

FIGS. 3A and 3B—Enzyme replacement therapy (ERT) in experimental SSADHD. (FIG. 3A) Aldh5a1−/− mouse survival-rate to day of life (DOL 30) as a function of enzyme replacement intervention. Purified human ALDH5A1 was administered daily (1 mg/kg/day), beginning at DOL 10, via i.p. injection. (FIG. 3B) The expression of GABA-related genes following ERT in a1 dh5a1−/− mice (fold change relative to aldh5a1+/+ mice; sagittal slices of 21 day old mice). Abbreviations: Gabra5, GABAA receptor subunit α5; Gabra6, α-6; Gabrb1, β-1; Gabrb3, β-3; Gabrd, δ; Gabre, ε; Gabrg1, γ-1; Gabrg2, γ-2; Gabrg3, γ-3; Gabrq, θ. Asterisked values represent directional correction of expression as a function of ERT.

FIGS. 4A and 4B—Metabolic measures in animals treated with ERT. The treatment scheme is described in the legend to FIG. 3. (FIG. 4A) Brain GHB content as a function of genotype (WT=wild-type, aldh5a1+/+ mice; MT=mutant, aldh5a1−/− mice; PBS, phosphate-buffered saline; SSADH, recombinant SSADH. (FIG. 4B) GABA in sera. Data depicted as mean±SD. Statistical analyses employed one-way ANOVA with post-hoc analysis (t test).

FIGS. 5A-5C—Intracellular chloride homeostasis, GABAergic neurotransmission and bumetanide in SSADHD (for abbreviations, see FIG. 1). (FIG. 5A) Schematic diagram of membrane ion transport and mechanism of action of bumetanide. (FIG. 5B) NKCC1 and KCC2 gene expression in the hypothalamus of aldh5a1+/+(n=5) and aldh5a1−/− (n=5) mice collected at postnatal day-of-life 20. Expression data were obtained using q-RT-PCR, validated pathway plates from Bio-Rad. (FIG. 5C) Time to sedation following acute dosing of 100 mg/kg bumetanide. Animals were evaluated at day of life (DOL) 20 and 24 for seizure events, and the time to immobilization (sedation) determined by visual recording using Noldus technology (see text). Bumetanide was administered intraperitoneally to the subject mice after 20 min. of initial observational recording, followed by an additional 20 minutes of recording. Total number of seizures was quantified in 5 min blocks of the total 40 min. recording period. Bumetanide was obtained from Enzo Life Sciences, Inc. (Farmingdale, N.Y., USA) and aseptically dissolved in DMSO vehicle. Vehicle-treated subjects did not show sedation. Aldh5a1−/− mice (mutant; MT) were significantly more resistant to the sedative effects of bumetanide as compared to aldh5a1+/+(wild-type; WT) mice, the latter showing almost instantaneous immobilization. During the active period in aldh5a1−/− mice prior to immobilization (˜3-8 min.), no seizure activity was noted. Additionally, this resistance increased significantly with age in aldh5a1−/− mice (t test, p<0.05). Abbreviations employed: n, number of animals studied; DOL, day of life; sec, seconds; min, minutes.

FIG. 6—Simplified schematic diagram of the roles of AMPK and mTORC1 in the regulation of autophagy. Both AMPK (adenosine monophosphate-activated protein kinase) and mTOR (mechanistic target of rapamycin) have key roles in the regulation of intracellular energetics, growth, and survival. Prkag1 and Prkag2 (protein kinase cAMP-activated γ subunits 1 and 2) represent components of the active AMPK trimeric structure. Conversely, RagB and D (ras-related GTP-binding proteins B and D) are involved in the regulation of mTORC1 function. Tsc1/2 (tuberous sclerosis proteins 1 and 2) are negative regulators of mTORC1. Abbreviations: HMG-CoA reductase, 3-hydroxy-3-methylglutaryl-coenzyme A reductase; ACC, acetyl-CoA carboxylase; ATP, adenosine triphosphate; AMP, adenosine monophosphate.

FIG. 7—Representative electrocorticographic recordings of seizure types in an aldh5a1−/− mouse. These data are representative of traces employed to generate the data of Table 2.

The figures herein are for illustrative purposes only and are not necessarily drawn to scale.

### DETAILED DESCRIPTION OF THE EXAMPLE EMBODIMENTS

### General Definitions

Unless defined otherwise, technical and scientific terms used herein have the same meaning as commonly understood by one of ordinary skill in the art to which this disclosure pertains. Definitions of common terms and techniques in molecular biology may be found in Molecular Cloning: A Laboratory Manual, 2nd edition (1989) (Sambrook, Fritsch, and Maniatis); Molecular Cloning: A Laboratory Manual, 4th edition (2012) (Green and Sambrook); Current Protocols in Molecular Biology (1987) (F. M. Ausubel et al. eds.); the series Methods in Enzymology (Academic Press, Inc.): PCR 2: A Practical Approach (1995) (M. J. MacPherson, B. D. Hames, and G. R. Taylor eds.): Antibodies, A Laboratory Manual (1988) (Harlow and Lane, eds.): Antibodies A Laboratory Manual, 2nd edition 2013 (E. A. Greenfield ed.); Animal Cell Culture (1987) (R. I. Freshney, ed.); Benjamin Lewin, Genes IX, published by Jones and Bartlet, 2008 (ISBN 0763752223); Kendrew et al. (eds.), The Encyclopedia of Molecular Biology, published by Blackwell Science Ltd., 1994 (ISBN 0632021829); Robert A. Meyers (ed.), Molecular Biology and Biotechnology: a Comprehensive Desk Reference, published by VCH Publishers, Inc., 1995 (ISBN 9780471185710); Singleton et al., Dictionary of Microbiology and Molecular Biology 2nd ed., J. Wiley & Sons (New York, N.Y. 1994), March, Advanced Organic Chemistry Reactions, Mechanisms and Structure 4th ed., John Wiley & Sons (New York, N.Y. 1992); and Marten H. Hofker and Jan van Deursen, Transgenic Mouse Methods and Protocols, 2nd edition (2011)

As used herein, the singular forms “a”, “an”, and “the” include both singular and plural referents unless the context clearly dictates otherwise.

The term “optional” or “optionally” means that the subsequent described event, circumstance or substituent may or may not occur, and that the description includes instances where the event or circumstance occurs and instances where it does not.

The recitation of numerical ranges by endpoints includes all numbers and fractions subsumed within the respective ranges, as well as the recited endpoints.

The terms “about” or “approximately” as used herein when referring to a measurable value such as a parameter, an amount, a temporal duration, and the like, are meant to encompass variations of and from the specified value, such as variations of +/−10% or less, +/−5% or less, +/−1% or less, and +1-0.1% or less of and from the specified value, insofar such variations are appropriate to perform in the disclosed invention. It is to be understood that the value to which the modifier “about” or “approximately” refers is itself also specifically, and preferably, disclosed.

As used herein, a “biological sample” may contain whole cells and/or live cells and/or cell debris. The biological sample may contain (or be derived from) a “bodily fluid”. The present invention encompasses embodiments wherein the bodily fluid is selected from amniotic fluid, aqueous humour, vitreous humour, bile, blood serum, breast milk, cerebrospinal fluid, cerumen (earwax), chyle, chyme, endolymph, perilymph, exudates, feces, female ejaculate, gastric acid, gastric juice, lymph, mucus (including nasal drainage and phlegm), pericardial fluid, peritoneal fluid, pleural fluid, pus, rheum, saliva, sebum (skin oil), semen, sputum, synovial fluid, sweat, tears, urine, vaginal secretion, vomit and mixtures of one or more thereof. Biological samples include cell cultures, bodily fluids, cell cultures from bodily fluids. Bodily fluids may be obtained from a mammal organism, for example by puncture, or other collecting or sampling procedures.

The terms “subject,” “individual,” and “patient” are used interchangeably herein to refer to a vertebrate, preferably a mammal, more preferably a human. Mammals include, but are not limited to, murines, simians, humans, farm animals, sport animals, and pets. Tissues, cells and their progeny of a biological entity obtained in vivo or cultured in vitro are also encompassed.

Various embodiments are described hereinafter. It should be noted that the specific embodiments are not intended as an exhaustive description or as a limitation to the broader aspects discussed herein. One aspect described in conjunction with a particular embodiment is not necessarily limited to that embodiment and can be practiced with any other embodiment(s). Reference throughout this specification to “one embodiment”, “an embodiment,” “an example embodiment,” means that a particular feature, structure or characteristic described in connection with the embodiment is included in at least one embodiment of the present invention. Thus, appearances of the phrases “in one embodiment,” “in an embodiment,” or “an example embodiment” in various places throughout this specification are not necessarily all referring to the same embodiment, but may. Furthermore, the particular features, structures or characteristics may be combined in any suitable manner, as would be apparent to a person skilled in the art from this disclosure, in one or more embodiments. Furthermore, while some embodiments described herein include some but not other features included in other embodiments, combinations of features of different embodiments are meant to be within the scope of the invention. For example, in the appended claims, any of the claimed embodiments can be used in any combination.

All publications, published patent documents, and patent applications cited herein are hereby incorporated by reference to the same extent as though each individual publication, published patent document, or patent application was specifically and individually indicated as being incorporated by reference.

### Compositions

In certain aspects, the invention comprises one or more compositions for treating SSADHD. In some embodiments, SSADHD may manifest through higher than normal levels circulating levels of metabolites. Such metabolites include, but are not necessarily limited to gamma-hydroxybutyric acid (GHB) and γ-aminobutyric acid (GABA).

As used in this context, to “treat” means to cure, ameliorate, stabilize, prevent, or reduce the severity of at least one symptom or a disease, pathological condition, or disorder. This term includes active treatment, that is, treatment directed specifically toward the improvement of a disease, pathological condition, or disorder, and also includes causal treatment, that is, treatment directed toward removal of the cause of the associated disease, pathological condition, or disorder. In addition, this term includes palliative treatment, that is, treatment designed for the relief of symptoms rather than the curing of the disease, pathological condition, or disorder; preventative treatment, that is, treatment directed to minimizing or partially or completely inhibiting the development of the associated disease, pathological condition, or disorder; and supportive treatment, that is, treatment employed to supplement another specific therapy directed toward the improvement of the associated disease, pathological condition, or disorder. It is understood that treatment, while intended to cure, ameliorate, stabilize, or prevent a disease, pathological condition, or disorder, need not actually result in the cure, amelioration, stabilization or prevention. The effects of treatment can be measured or assessed as described herein and as known in the art as is suitable for the disease, pathological condition, or disorder involved. Such measurements and assessments can be made in qualitative and/or quantitative terms. Thus, for example, characteristics or features of a disease, pathological condition, or disorder and/or symptoms of a disease, pathological condition, or disorder can be reduced to any effect or to any amount.

The term “in need of treatment” as used herein refers to a judgment made by a caregiver (e.g. physician, nurse, nurse practitioner, or individual in the case of humans; veterinarian in the case of animals, including non-human animals) that a subject requires or will benefit from treatment. This judgment is made based on a variety of factors that are in the realm of a caregiver's experience, but that include the knowledge that the subject is ill, or will be ill, as the result of a condition that is treatable by the compositions and therapeutic agents described herein.

As used herein, the term “functional” or “functional SSADH enzyme” refers to an enzyme that functions normally, as opposed to an enzyme that originates from a gene with a mutation that renders the enzyme non-existent, deficient, defective or non-functional. In some embodiments, a functional SSADH enzyme would be a fully operational enzyme, such as an enzyme found in a healthy individual harboring the wild type gene that codes for SSADH.

Succinic semialdehyde dehydrogenase (SSADH) is an enzyme in the GABA degradation pathway that converts succinic semialdehyde into succinate, an essential component of the Krebs cycle. In the case of SSADH deficiency, succinic semialdehyde (SSA), the final intermediate of the GABA degradation pathway, accumulates and cannot be oxidized to succinic acid. As a result, SSA is reduced to GHB by gamma-hydroxybutyric dehydrogenase. This leads to elevated circulating levels of both GABA and GHB.

As used herein, a “vector” is a tool that allows or facilitates the transfer of an entity from one environment to another. It is a replicon, such as a plasmid, phage, or cosmid, into which another DNA segment may be inserted so as to bring about the replication of the inserted segment. Generally, a vector is capable of replication when associated with the proper control elements. In general, the term “vector” refers to a nucleic acid molecule capable of transporting another nucleic acid to which it has been linked. Vectors include, but are not limited to, nucleic acid molecules that are single-stranded, double-stranded, or partially double-stranded; nucleic acid molecules that comprise one or more free ends, no free ends (e.g. circular); nucleic acid molecules that comprise DNA, RNA, or both; and other varieties of polynucleotides known in the art. One type of vector is a “plasmid,” which refers to a circular double stranded DNA loop into which additional DNA segments can be inserted, such as by standard molecular cloning techniques. Another type of vector is a viral vector, wherein virally-derived DNA or RNA sequences are present in the vector for packaging into a virus (e.g. retroviruses, replication defective retroviruses, adenoviruses, replication defective adenoviruses, and adeno-associated viruses (AAVs)). Viral vectors also include polynucleotides carried by a virus for transfection into a host cell. Certain vectors are capable of autonomous replication in a host cell into which they are introduced (e.g. bacterial vectors having a bacterial origin of replication and episomal mammalian vectors). Other vectors (e.g., non-episomal mammalian vectors) are integrated into the genome of a host cell upon introduction into the host cell, and thereby are replicated along with the host genome. Moreover, certain vectors are capable of directing the expression of genes to which they are operatively-linked. Such vectors are referred to herein as “expression vectors.” Common expression vectors of utility in recombinant DNA techniques are often in the form of plasmids.

Recombinant expression vectors can comprise a nucleic acid of the invention in a form suitable for expression of the nucleic acid in a host cell, which means that the recombinant expression vectors include one or more regulatory elements, which may be selected on the basis of the host cells to be used for expression, that is operably linked to the nucleic acid sequence to be expressed. With regards to recombination and cloning methods, mention is made of U.S. patent application Ser. No. 10/815,730, published Sep. 2, 2004 as US 2004-0171156 A1, the contents of which are herein incorporated by reference in their entirety.

In some embodiments, the vector may be a retroviral vector. Retroviral vectors may include, but are not necessarily limited to, lentiviral vectors. Lentiviruses are complex retroviruses that have the ability to infect and express their genes in both mitotic and post-mitotic cells. The most commonly known lentivirus is the human immunodeficiency virus (HIV), which uses the envelope glycoproteins of other viruses to target a broad range of cell types.

Lentiviruses may be prepared by any method known in the art. One exemplary method may include cloning the gene of interest into a plasmid which contains a lentiviral transfer plasmid backbone. Cells may be seeded at low passage (p=5) in a T-75 flask to 50% confluence the day before transfection in DMEM with 10% fetal bovine serum and without antibiotics. After 20 hours, media can be changed to OptiMEM (serum-free) media and transfection may be done 4 hours later. Cells can be transfected with 10 μg of lentiviral transfer plasmid and the following packaging plasmids: 5 μg of pMD2.G (VSV-g pseudotype), and 7.5 ug of psPAX2 (gag/pol/rev/tat). Transfection may be done in 4 mL OptiMEM with a cationic lipid delivery agent (50 uL Lipofectamine 2000 and 100 ul Plus reagent). After 6 hours, the media can be changed to antibiotic-free DMEM with 10% fetal bovine serum. These methods use serum during cell culture, but serum-free methods are preferred.

Lentivirus may be purified as follows. Viral supernatants can be harvested after 48 hours. Supernatants can first be cleared of debris and filtered through a 0.45 um low protein binding (PVDF) filter. They are then spun in an ultracentrifuge for 2 hours at 24,000 rpm. Viral pellets are resuspended in 50 ul of DMEM overnight at 4 C. They are then aliquoted and immediately frozen at −80° C.

In another embodiment, minimal non-primate lentiviral vectors based on the equine infectious anemia virus (EIAV) are also contemplated, especially for ocular gene therapy (see, e.g., Balagaan, J Gene Med 2006; 8: 275-285). In another embodiment, RetinoStat®, an equine infectious anemia virus-based lentiviral gene therapy vector that expresses angiostatic proteins endostatin and angiostatin that is delivered via a subretinal injection for the treatment of the web form of age-related macular degeneration is also contemplated (see, e.g., Binley et a1., HUMAN GENE THERAPY 23:980-991 (September 2012)) and this vector may be modified as needed to be suitable for the present invention.

In another embodiment, self-inactivating lentiviral vectors with an siRNA targeting a common exon shared by HIV tat/rev, a nucleolar-localizing TAR decoy, and an anti-CCR5-specific hammerhead ribozyme (see, e.g., DiGiusto et al. (2010) Sci Transl Med 2:36ra43) may be used/and or adapted to the system of the present invention. A minimum of 2.5×106 CD34+ cells per kilogram patient weight may be collected and prestimulated for 16 to 20 hours in X-VIVO 15 medium (Lonza) containing 2 μmon-glutamine, stem cell factor (100 ng/ml), Flt-3 ligand (Flt-3L) (100 ng/ml), and thrombopoietin (10 ng/ml) (CellGenix) at a density of 2×106 cells/ml. Prestimulated cells may be transduced with lentiviral at a multiplicity of infection of 5 for 16 to 24 hours in 75-cm2 tissue culture flasks coated with fibronectin (25 mg/cm2) (RetroNectin, Takara Bio Inc.).

Lentiviral vectors have been disclosed as in the treatment for Parkinson's Disease, see, e.g., US Patent Publication No. 20120295960 and U.S. Pat. Nos. 7,303,910 and 7,351,585. Lentiviral vectors have also been disclosed for the treatment of ocular diseases, see e.g., US Patent Publication Nos. 20060281180, 20090007284, US20110117189; US20090017543; US20070054961, US20100317109. Lentiviral vectors have also been disclosed for delivery to the brain, see, e.g., US Patent Publication Nos. US20110293571; US20110293571, US20040013648, US20070025970, US20090111106 and U.S. Pat. No. 7,259,015.

In specific embodiments, the targeting vector is a viral vector, such as including, but not necessarily limited to, a retroviral vector, adenoviral vector, or adeno-associated viral vector. In specific embodiments, the retroviral vector is a lentiviral vector.

For example, for adeno-associated viral vectors (AAV), the route of administration, formulation and dose can be as in U.S. Pat. No. 8,454,972 and as in clinical trials involving adeno-associated viral vector. For adenovirus, the route of administration, formulation and dose can be as in U.S. Pat. No. 8,404,658 and as in clinical trials involving adenovirus. For plasmid delivery, the route of administration, formulation and dose can be as in U.S. Pat. No. 5,846,946 and as in clinical studies involving plasmids. Doses may be based on or extrapolated to an average 70 kg individual (e.g. a male adult human), and can be adjusted for patients, subjects, mammals of different weight and species. Frequency of administration is within the ambit of the medical or veterinary practitioner (e.g., physician, veterinarian), depending on usual factors including the age, sex, general health, other conditions of the patient or subject and the particular condition or symptoms being addressed. The viral vectors can be injected into the tissue of interest. Cell-type specific expression can be driven by a cell-type specific promoter. For example, liver-specific expression might use the Albumin promoter and neuron-specific expression (e.g. for targeting CNS disorders) might use the Synapsin I promoter.

In terms of in vivo delivery, AAV is advantageous over other viral vectors for a couple of reasons. It has low toxicity (this may be due to the purification method not requiring ultra centrifugation of cell particles that can activate the immune response) and it has a low probability of causing insertional mutagenesis because it doesn't integrate into the host genome. As to AAV, the AAV can be AAV1, AAV2, AAV5 or any combination thereof. One can select the AAV of the AAV with regard to the cells to be targeted; e.g., one can select AAV serotypes 1, 2, 5 or a hybrid capsid AAV1, AAV2, AAV5 or any combination thereof for targeting brain or neuronal cells; and one can select AAV4 for targeting cardiac tissue. AAV8 is useful for delivery to the liver.

In some aspects or embodiments, a composition comprising a delivery particle formulation may be used. In some embodiments, the delivery particle comprises a lipid-based particle, optionally a lipid nanoparticle, or cationic lipid and optionally biodegradable polymer. In some embodiments, the cationic lipid comprises 1,2-dioleoyl-3-trimethylammonium-propane (DOTAP). In some embodiments, the hydrophilic polymer comprises ethylene glycol or polyethylene glycol. In some embodiments, the delivery particle further comprises a lipoprotein, preferably cholesterol. In some embodiments, the delivery particles are less than 500 nm in diameter, optionally less than 250 nm in diameter, optionally less than 100 nm in diameter, optionally about 35 nm to about 60 nm in diameter.

Several types of particle delivery systems and/or formulations are known to be useful in a diverse spectrum of biomedical applications. In general, a particle is defined as a small object that behaves as a whole unit with respect to its transport and properties. Particles are further classified according to diameter. Coarse particles cover a range between 2,500 and 10,000 nanometers. Fine particles are sized between 100 and 2,500 nanometers. Ultrafine particles, or nanoparticles, are generally between 1 and 100 nanometers in size. The basis of the 100-nm limit is the fact that novel properties that differentiate particles from the bulk material typically develop at a critical length scale of under 100 nm.

As used herein, a particle delivery system/formulation is defined as any biological delivery system/formulation which includes a particle in accordance with the present invention. A particle in accordance with the present invention is any entity having a greatest dimension (e.g. diameter) of less than 100 microns (μm). In some embodiments, inventive particles have a greatest dimension of less than 10 μm. In some embodiments, inventive particles have a greatest dimension of less than 2000 nanometers (nm). In some embodiments, inventive particles have a greatest dimension of less than 1000 nanometers (nm). In some embodiments, inventive particles have a greatest dimension of less than 900 nm, 800 nm, 700 nm, 600 nm, 500 nm, 400 nm, 300 nm, 200 nm, or 100 nm. Typically, inventive particles have a greatest dimension (e.g., diameter) of 500 nm or less. In some embodiments, inventive particles have a greatest dimension (e.g., diameter) of 250 nm or less. In some embodiments, inventive particles have a greatest dimension (e.g., diameter) of 200 nm or less. In some embodiments, inventive particles have a greatest dimension (e.g., diameter) of 150 nm or less. In some embodiments, inventive particles have a greatest dimension (e.g., diameter) of 100 nm or less. Smaller particles, e.g., having a greatest dimension of 50 nm or less are used in some embodiments of the invention. In some embodiments, inventive particles have a greatest dimension ranging between 25 nm and 200 nm.

In general, a “nanoparticle” refers to any particle having a diameter of less than 1000 nm. In certain preferred embodiments, nanoparticles of the invention have a greatest dimension (e.g., diameter) of 500 nm or less. In other preferred embodiments, nanoparticles of the invention have a greatest dimension ranging between 25 nm and 200 nm. In other preferred embodiments, nanoparticles of the invention have a greatest dimension of 100 nm or less. In other preferred embodiments, nanoparticles of the invention have a greatest dimension ranging between 35 nm and 60 nm. It will be appreciated that reference made herein to particles or nanoparticles can be interchangeable, where appropriate.

It will be understood that the size of the particle will differ depending as to whether it is measured before or after loading. Accordingly, in particular embodiments, the term “nanoparticles” may apply only to the particles pre loading.

Nanoparticles encompassed in the present invention may be provided in different forms, e.g., as solid nanoparticles (e.g., metal such as silver, gold, iron, titanium), non-metal, lipid-based solids, polymers), suspensions of nanoparticles, or combinations thereof. Metal, dielectric, and semiconductor nanoparticles may be prepared, as well as hybrid structures (e.g., core-shell nanoparticles). Nanoparticles made of semiconducting material may also be labeled quantum dots if they are small enough (typically sub 10 nm) that quantization of electronic energy levels occurs. Such nanoscale particles are used in biomedical applications as drug carriers or imaging agents and may be adapted for similar purposes in the present invention.

Semi-solid and soft nanoparticles have been manufactured, and are within the scope of the present invention. A prototype nanoparticle of semi-solid nature is the liposome. Various types of liposome nanoparticles are currently used clinically as delivery systems for anticancer drugs and vaccines. Nanoparticles with one half hydrophilic and the other half hydrophobic are termed Janus particles and are particularly effective for stabilizing emulsions. They can self-assemble at water/oil interfaces and act as solid surfactants.

Particle characterization (including e.g., characterizing morphology, dimension, etc.) is done using a variety of different techniques. Common techniques are electron microscopy (TEM, SEM), atomic force microscopy (AFM), dynamic light scattering (DLS), X-ray photoelectron spectroscopy (XPS), powder X-ray diffraction (XRD), Fourier transform infrared spectroscopy (FTIR), matrix-assisted laser desorption/ionization time-of-flight mass spectrometry (MALDI-TOF), ultraviolet-visible spectroscopy, dual polarization interferometry and nuclear magnetic resonance (NMR). Characterization (dimension measurements) may be made as to native particles (i.e., preloading) or after loading of the cargo (herein cargo refers to e.g., a gene encoding a functional SSADH enzyme, a drug, or any combination thereof, and may include additional carriers and/or excipients) to provide particles of an optimal size for delivery for any in vitro, ex vivo and/or in vivo application of the present invention. In certain preferred embodiments, particle dimension (e.g., diameter) characterization is based on measurements using dynamic laser scattering (DLS). Mention is made of U.S. Pat. Nos. 8,709,843; 6,007,845; 5,855,913; 5,985,309; 5,543,158; and the publication by James E. Dahlman and Carmen Barnes et al. Nature Nanotechnology (2014) published online 11 May 2014, doi:10.1038/nnano.2014.84, concerning particles, methods of making and using them and measurements thereof.

Particles delivery systems within the scope of the present invention may be provided in any form, including but not limited to solid, semi-solid, emulsion, or colloidal particles. As such any of the delivery systems described herein, including but not limited to, e.g., lipid-based systems, liposomes, micelles, microvesicles, exosomes, or gene gun may be provided as particle delivery systems within the scope of the present invention.

In another embodiment, lipid nanoparticles (LNPs) are contemplated. An antitransthyretin small interfering RNA has been encapsulated in lipid nanoparticles and delivered to humans (see, e.g., Coelho et al., N Engl J Med 2013; 369:819-29), and such a system may be adapted and applied to the system of the present invention. Doses of about 0.01 to about 1 mg per kg of body weight administered intravenously are contemplated. Medications to reduce the risk of infusion-related reactions are contemplated, such as dexamethasone, acetampinophen, diphenhydramine or cetirizine, and ranitidine are contemplated. Multiple doses of about 0.3 mg per kilogram every 4 weeks for five doses are also contemplated.

Zhu et al. (US20140348900) provides for a process for preparing liposomes, lipid discs, and other lipid nanoparticles using a multi-port manifold, wherein the lipid solution stream, containing an organic solvent, is mixed with two or more streams of aqueous solution (e.g., buffer). In some aspects, at least some of the streams of the lipid and aqueous solutions are not directly opposite of each other. Thus, the process does not require dilution of the organic solvent as an additional step. In some embodiments, one of the solutions may also contain an active pharmaceutical ingredient (API). This invention provides a robust process of liposome manufacturing with different lipid formulations and different payloads. Particle size, morphology, and the manufacturing scale can be controlled by altering the port size and number of the manifold ports, and by selecting the flow rate or flow velocity of the lipid and aqueous solutions.

U.S. Pat. No. 8,709,843, incorporated herein by reference, provides a drug delivery system for targeted delivery of therapeutic agent-containing particles to tissues, cells, and intracellular compartments. The invention provides targeted particles comprising polymer conjugated to a surfactant, hydrophilic polymer or lipid.

The lipid or lipid-like compounds described above include the compounds themselves, as well as their salts and solvates, if applicable. A salt, for example, can be formed between an anion and a positively charged group (e.g., amino) on a lipid-like compound. Suitable anions include chloride, bromide, iodide, sulfate, nitrate, phosphate, citrate, methanesulfonate, trifluoroacetate, acetate, malate, tosylate, tartrate, fumurate, glutamate, glucuronate, lactate, glutarate, and maleate. Likewise, a salt can also be formed between a cation and a negatively charged group (e.g., carboxylate) on a lipid-like compound. Suitable cations include sodium ion, potassium ion, magnesium ion, calcium ion, and an ammonium cation such as tetramethylammonium ion. The lipid-like compounds also include those salts containing quaternary nitrogen atoms. A solvate refers to a complex formed between a lipid-like compound and a pharmaceutically acceptable solvent. Examples of pharmaceutically acceptable solvents include water, ethanol, isopropanol, ethyl acetate, acetic acid, and ethanolamine.

Delivery or administration according to the invention can be performed with liposomes. Liposomes are spherical vesicle structures composed of a uni- or multilamellar lipid bilayer surrounding internal aqueous compartments and a relatively impermeable outer lipophilic phospholipid bilayer. Liposomes have gained considerable attention as drug delivery carriers because they are biocompatible, nontoxic, can deliver both hydrophilic and lipophilic drug molecules, protect their cargo from degradation by plasma enzymes, and transport their load across biological membranes and the blood brain barrier (BBB) (see, e.g., Spuch and Navarro, Journal of Drug Delivery, vol. 2011, Article ID 469679, 12 pages, 2011. doi: 10.1155/2011/469679 for review).

Liposomes can be made from several different types of lipids; however, phospholipids are most commonly used to generate liposomes as drug carriers. Although liposome formation is spontaneous when a lipid film is mixed with an aqueous solution, it can also be expedited by applying force in the form of shaking by using a homogenizer, sonicator, or an extrusion apparatus (see, e.g., Spuch and Navarro, Journal of Drug Delivery, vol. 2011, Article ID 469679, 12 pages, 2011. doi:10.1155/2011/469679 for review).

Several other additives may be added to liposomes in order to modify their structure and properties. For instance, either cholesterol or sphingomyelin may be added to the liposomal mixture in order to help stabilize the liposomal structure and to prevent the leakage of the liposomal inner cargo. Further, liposomes are prepared from hydrogenated egg phosphatidylcholine or egg phosphatidylcholine, cholesterol, and dicetyl phosphate, and their mean vesicle sizes were adjusted to about 50 and 100 nm. (see, e.g., Spuch and Navarro, Journal of Drug Delivery, vol. 2011, Article ID 469679, 12 pages, 2011. doi:10.1155/2011/469679 for review).

A liposome formulation may be mainly comprised of natural phospholipids and lipids such as 1,2-di stearoryl-sn-glycero-3-phosphatidyl choline (DSPC), sphingomyelin, egg phosphatidylcholines and monosialoganglioside. Since this formulation is made up of phospholipids only, liposomal formulations have encountered many challenges, one of the ones being the instability in plasma. Several attempts to overcome these challenges have been made, specifically in the manipulation of the lipid membrane. One of these attempts focused on the manipulation of cholesterol. Addition of cholesterol to conventional formulations reduces rapid release of the encapsulated bioactive compound into the plasma or 1,2-dioleoyl-sn-glycero-3-phosphoethanolamine (DOPE) increases the stability (see, e.g., Spuch and Navarro, Journal of Drug Delivery, vol. 2011, Article ID 469679, 12 pages, 2011. doi:10.1155/2011/469679 for review).

Specific targeting vectors can be developed that target antigens on specific cells. Upon local or systemic introduction, these vectors can circulate and home to specific cells. Targeting of specific cells and tissues would greatly enhance the safety of gene therapeutic applications. Inappropriate expression due to inadvertent infection of irrelevant cells or tissues is one cause for concern in gene therapy applications. Accordingly, targeting to specific cells would lessen the possibility of adverse side effects. In some embodiments, such target cells may include, but are not necessarily limited to, liver cells, lymph, blood, plasma, cerebrospinal fluid, pancreas, nephron, glial cells, astroglia, oligodendrocytes, neurons, astrocytes, hepatocytes, white blood cells, monocytes, leukocytes, spleen, platelets, gonads, ovaries, eye, retinal pigment epithelia, amacrine cells, bipolar cells, vitreous humor, aqueous humor, retina, lens. In specific embodiments, the target cells are liver cells.

Within a recombinant expression vector, “operably linked” is intended to mean that the nucleotide sequence or gene of interest is linked or complexed to the regulatory element(s) or to the targeting vector in a manner that allows for expression of the nucleotide sequence or gene (e.g., in an in vitro transcription/translation system or in a host cell when the vector is introduced into the host cell).

In some embodiments, the invention comprises a composition for treating SSADHD comprising a gene encoding a functional SSADH enzyme operably linked to a targeting vector. In specific embodiments, the gene may be ALDH5A1. The ALDH5A1 gene belongs to the aldehyde dehydrogenase family of proteins. The gene encodes a mitochondrial NAD⁺-dependent succinic semialdehyde dehydrogenase (SSADH). As described elsewhere herein, a deficiency in this enzyme is a rare inborn error in the metabolism of the neurotransmitter GABA. In response to the defect, physiologic fluids from patients accumulate GHB, a compound with numerous neuromodulatory properties.

In some embodiments, ALDH5A1 may be produced in bacterial cell lines, as described in the examples. In some embodiments, ALDH5A1 may be produced in a number of bacterial and mammalian cells, including, but not necessarily limited to, HEK, yeast, and CHO cells.

The presence of a functional SSADH enzyme may lead to lower levels of circulating metabolites. Such circulation metabolites may include, but are not necessarily limited to, GHB and GABA. In specific embodiments, a functional SSADH enzyme lowers the levels of circulating GHB and GABA.

In some embodiments, the composition does not cross the blood brain barrier. The blood brain barrier is a highly selective semipermeable border that separates the circulating blood from the brain and extracellular fluid in the central nervous system. The blood brain barrier is formed by endothelial cells of the capillary wall, astrocyte end-feet ensheathing the capillary, and pericytes embedded in the capillary basement membrane. This allows the passage of water, some gases, and lipid-soluble molecules by passive diffusion, as well as the selective transport of molecules such as glucose and amino acids that are crucial to neuron function. The blood brain barrier restricts the diffusion of solutes in the blood (e.g. bacteria) and large or hydrophilic molecules into the cerebrospinal fluid, while allowing the diffusion of hydrophobic molecules (oxygen, carbon dioxide, hormones) and small polar molecules. Cells of the barrier actively transport metabolic products such as glucose across the barrier using specific transport proteins.

### Methods of Treatment

Also envisioned within the scope of the invention are methods of treating SSADHD in a subject in need thereof. Such methods may comprise administering a therapeutically effective amount of any of the compositions described herein. As described elsewhere herein, to “treat” means to cure, ameliorate, stabilize, prevent, or reduce the severity of at least one symptom or a disease, pathological condition, or disorder.

In specific embodiments, the subject has SSADHD. In specific embodiments, the subject has increased circulating levels of metabolites. The terms “high,” “higher,” “increased,” “elevated,” or “elevation” refer to increases above basal levels, e.g., as compared to a control. The terms “low,” “lower,” “reduced,” or “reduction refer to decreases below basal levels, e.g., as compared to a control.

The term “control” refers to any reference standard suitable to provide a comparison to the expression products in the test sample. In one embodiment, the control comprises obtaining a “control sample” from which expression product levels are detected and compared to the expression product levels from the test sample. Such a control sample may comprise any suitable sample, including but not limited to a sample from a control patient (can be stored sample or previous sample measurement) with a known outcome; normal tissue, fluid, or cells isolated from a subject, such as a normal patient or the patient having a condition of interest.

The term “altered amount” or “altered level” refers to increased or decreased copy number (e.g., germline and/or somatic) of a metabolite or biomarker nucleic acid, as compared to the expression level or copy number of the metabolite or biomarker nucleic acid in a control sample. The term “altered amount” of a biomarker also includes an increased or decreased protein level of a biomarker protein in a sample, as compared to the corresponding protein level in a normal, control sample. Furthermore, an altered amount of a biomarker protein may be determined by detecting posttranslational modification such as methylation status of the marker, which may affect the expression or activity of the biomarker protein.

The amount of a metabolite or biomarker in a subject is “significantly” higher or lower than the normal amount of the metabolite or biomarker, if the amount of the biomarker is greater or less, respectively, than the normal or control level by an amount greater than the standard error of the assay employed to assess amount, and preferably at least 20%, 30%, 40%, 50%, 60%, 70%, 80%, 90%, 100%, 150%, 200%, 300%, 350%, 400%, 500%, 600%, 700%, 800%, 900%, 1000% or than that amount. Alternatively, the amount of the biomarker in the subject can be considered “significantly” higher or lower than the normal and/or control amount if the amount is at least about two, and preferably at least about 5%, 10%, 15%, 20%, 25%, 30%, 35%, 40%, 45%, 50%, 55%, 60%, 65%, 70%, 75%, 80%, 85%, 90%, 95%, 100%, 105%, 110%, 115%, 120%, 125%, 130%, 135%, 140%, 145%, 150%, 155%, 160%, 165%, 170%, 175%, 180%, 185%, 190%, 195%, two times, three times, four times, five times, or more, or any range in between, such as 5%-100%, higher or lower, respectively, than the normal and/or control amount of the biomarker. Such significant modulation values can be applied to any metric described herein, such as altered level of expression, altered activity, changes in cancer cell hyperproliferative growth, changes in cancer cell death, changes in biomarker inhibition, changes in test agent binding, and the like.

The term “altered level of expression” of a marker refers to an expression level or copy number of a marker in a test sample e.g., a sample derived from a subject suffering from cancer, that is greater or less than the standard error of the assay employed to assess expression or copy number, and is preferably at least twice, and more preferably three, four, five or ten or more times the expression level or copy number of the marker or chromosomal region in a control sample (e.g., sample from a healthy subject not having the associated disease) and preferably, the average expression level or copy number of the marker or chromosomal region in several control samples. The altered level of expression is greater or less than the standard error of the assay employed to assess expression or copy number, and is preferably at least twice, and more preferably three, four, five or ten or more times the expression level or copy number of the marker in a control sample (e.g., sample from a healthy subject not having the associated disease) and preferably, the average expression level or copy number of the marker in several control samples.

The term “altered activity” of a marker refers to an activity of a marker which is increased or decreased in a disease state, e.g., in a cancer sample, as compared to the activity of the marker in a normal, control sample. Altered activity of a marker may be the result of, for example, altered expression of the marker, altered protein level of the marker, altered structure of the marker, or, e.g., an altered interaction with other proteins involved in the same or different pathway as the marker, or altered interaction with transcriptional activators or inhibitors.

The “amount” of a metabolite or marker, e.g., expression or copy number of a metabolite or marker, or protein level of a marker, in a subject is “significantly” higher or lower than the normal amount of a marker, if the amount of the marker is greater or less, respectively, than the normal level by an amount greater than the standard error of the assay employed to assess amount, and preferably at least twice, and more preferably three, four, five, ten or more times that amount. Alternately, the amount of the marker in the subject can be considered “significantly” higher or lower than the normal amount if the amount is at least about two, and preferably at least about three, four, or five times, higher or lower, respectively, than the normal amount of the marker.

In specific embodiments, the subject has increased levels of circulating metabolites. Such metabolites may include alcohols, amino acids, nucleotides, antioxidants, organic acids, polyols, and vitamins. In specific embodiments, the metabolites described herein include, but are not necessarily limited to, GHB, GABA, or both. In specific embodiments, subjects with a SSADHD condition exhibit increased, or higher than normal circulating levels of GHB, GABA, or both.

In some embodiments, the vector, e.g., plasmid or viral vector is delivered to the tissue of interest by, for example intravenous, intradermal, transdermal, subcutaneous, intramuscular, intraperitoneal, intrarectal, intraarterial, intralymphatic, intrathecal, intratracheal, intranasal, oral, mucosal, or other delivery methods. The method of delivery may depend on whether local or systemic treatment is desired, and on the area to be treated. Parenteral administration, if used, is generally characterized by injection. Injectables can be prepared in conventional forms, either as liquid solutions or suspensions, solid forms suitable for solution or suspension in liquid prior to injection, or as emulsions.

Such delivery may be either via a single dose, or multiple doses. One skilled in the art understands that the actual dosage to be delivered herein may vary greatly depending upon a variety of factors, such as the vector choice, the target cell, organism, or tissue, the general condition of the subject to be treated, the degree of transformation/modification sought, the administration route, the administration mode, the type of transformation/modification sought, etc.

Such a dosage may further contain, for example, a carrier (water, saline, ethanol, glycerol, lactose, sucrose, calcium phosphate, gelatin, dextran, agar, pectin, peanut oil, sesame oil, etc.), a diluent, a pharmaceutically-acceptable carrier (e.g., phosphate-buffered saline), a pharmaceutically-acceptable excipient, and/or other compounds known in the art. The dosage may further contain one or more pharmaceutically acceptable salts such as, for example, a mineral acid salt such as a hydrochloride, a hydrobromide, a phosphate, a sulfate, etc.; and the salts of organic acids such as acetates, propionates, malonates, benzoates, etc. Additionally, auxiliary substances, such as wetting or emulsifying agents, pH buffering substances, gels or gelling materials, flavorings, colorants, microspheres, polymers, suspension agents, etc. may also be present herein. In addition, one or more other conventional pharmaceutical ingredients, such as preservatives, humectants, suspending agents, surfactants, antioxidants, anticaking agents, fillers, chelating agents, coating agents, chemical stabilizers, etc. may also be present, especially if the dosage form is a reconstitutable form. Suitable exemplary ingredients include microcrystalline cellulose, carboxymethylcellulose sodium, polysorbate 80, phenylethyl alcohol, chlorobutanol, potassium sorbate, sorbic acid, sulfur dioxide, propyl gallate, the parabens, ethyl vanillin, glycerin, phenol, parachlorophenol, gelatin, albumin and a combination thereof. A thorough discussion of pharmaceutically acceptable excipients is available in REMINGTON'S PHARMACEUTICAL SCIENCES (Mack Pub. Co., N.J. 1991) which is incorporated by reference herein.

In an embodiment herein the delivery is via an adenovirus, which may be at a single booster dose containing at least 1×105 particles (also referred to as particle units, pu) of adenoviral vector. In an embodiment herein, the dose preferably is at least about 1×106 particles (for example, about 1×106-1×1012 particles), more preferably at least about 1×107 particles, more preferably at least about 1×108 particles (e.g., about 1×108-1×1011 particles or about 1×108-1×1012 particles), and most preferably at least about 1×100 particles (e.g., about 1×109-1×1010 particles or about 1×109-1×1012 particles), or even at least about 1×1010 particles (e.g., about 1×1010-1×1012 particles) of the adenoviral vector. Alternatively, the dose comprises no more than about 1×1014 particles, preferably no more than about 1×1013 particles, even more preferably no more than about 1×1012 particles, even more preferably no more than about 1×1011 particles, and most preferably no more than about 1×1010 particles (e.g., no more than about 1×109 articles). Thus, the dose may contain a single dose of adenoviral vector with, for example, about 1×106 particle units (pu), about 2×106 pu, about 4×106 pu, about 1×107 pu, about 2×107 pu, about 4×107 pu, about 1×108 pu, about 2×108 pu, about 4×108 pu, about 1×109 pu, about 2×109 pu, about 4×109 pu, about 1×1010 pu, about 2×1010 pu, about 4×1010 pu, about 1×1011 pu, about 2×1011 pu, about 4×1011 pu, about 1×1012 pu, about 2×1012 pu, or about 4×1012 pu of adenoviral vector. See, for example, the adenoviral vectors in U.S. Pat. No. 8,454,972 B2 to Nabel, et. al., granted on Jun. 4, 2013; incorporated by reference herein, and the dosages at col 29, lines 36-58 thereof. In an embodiment herein, the adenovirus is delivered via multiple doses.

In an embodiment herein, the delivery is via an AAV. A therapeutically effective dosage for in vivo delivery of the AAV to a human is believed to be in the range of from about 20 to about 50 ml of saline solution containing from about 1×1010 to about 1×1010 functional AAV/ml solution. The dosage may be adjusted to balance the therapeutic benefit against any side effects. In an embodiment herein, the AAV dose is generally in the range of concentrations of from about 1×105 to 1×1050 genomes AAV, from about 1×108 to 1×1020 genomes AAV, from about 1×1010 to about 1×1016 genomes, or about 1×1011 to about 1×1016 genomes AAV. A human dosage may be about 1×1013 genomes AAV. Such concentrations may be delivered in from about 0.001 ml to about 100 ml, about 0.05 to about 50 ml, or about 10 to about 25 ml of a carrier solution. Other effective dosages can be readily established by one of ordinary skill in the art through routine trials establishing dose response curves. See, for example, U.S. Pat. No. 8,404,658 B2 to Hajjar, et al., granted on Mar. 26, 2013, at col. 27, lines 45-60.

In an embodiment herein the delivery is via a plasmid. In such plasmid compositions, the dosage should be a sufficient amount of plasmid to elicit a response. For instance, suitable quantities of plasmid DNA in plasmid compositions can be from about 0.1 to about 2 mg, or from about 1 μg to about 10 μg per 70 kg individual.

The doses herein are based on an average 70 kg individual. The frequency of administration is within the ambit of the medical or veterinary practitioner (e.g., physician, veterinarian), or scientist skilled in the art. It is also noted that mice used in experiments are typically about 20 g and from mice experiments one can scale up to a 70 kg individual.

The dosage used for the compositions provided herein include dosages for repeated administration or repeat dosing. In particular embodiments, the administration is repeated within a period of several weeks, months, or years. Suitable assays can be performed to obtain an optimal dosage regime. Repeated administration can allow the use of lower dosage, which can positively affect off-target modifications.

By the term “effective amount” or “therapeutically effective amount” of a compound, composition, or drug as provided herein, is meant a nontoxic but sufficient amount of the composition to provide the desired result. The exact amount required will vary from subject to subject, depending on the species, age, and general condition of the subject, the severity of the disease that is being treated, the particular composition used, its mode of administration, and the like. Thus, it is not possible to specify and exact “effective amount.” However, an appropriate effective amount can be determined by one of ordinary skill in the art using only routine experimentation.

In some embodiments, the therapeutically effective amount comprises a range of 1-10,000 μg functional SSADH enzyme per kg of body weight per day. In some embodiments, the composition may be administered once per week, bi-weekly, or once a month.

In some embodiments, the invention comprises a method of treating SSADHD in a subject in need thereof, comprising administering to the subject therapeutically effective amounts of a composition comprising a gene encoding a functional SSADH enzyme operably linked to a targeting vector as described herein, and one or more mTOR inhibitors, a GABA-T inhibitor, or a combination thereof.

mTOR (mammalian target of rapamycin) is a kinase that is a member of the phosphatidylinositol 3-kinase-related kinase family of protein kinases. mTOR links with other proteins and serves as a core component of two distinct protein complexes, mTOR complex 1 and mTOR complex 2, which regulate different cellular processes. In particular, as a core component of both complexes, mTOR functions as a serine/threonine protein kinase that regulates cell growth, cell proliferation, cell motility, cell survival, protein synthesis, autophagy, and transcription. mTOR inhibitors are a class of drugs that inhibit the mTOR kinase.

The term “inhibit” or “downregulate” includes the decrease, limitation, or blockage, of, for example a particular action, function, or interaction. A biological function, such as the function of a protein, is inhibited if it is decreased as compared to a reference state, such as a control like a wild-type state. Such inhibition or deficiency can be induced, such as by application of agent at a particular time and/or place, or can be constitutive, such as by a heritable mutation. Such inhibition or deficiency can also be partial or complete (e.g., essentially no measurable activity in comparison to a reference state, such as a control like a wild-type state). Essentially complete inhibition or deficiency is referred to as blocked. The term “promote” or “upregulate” has the opposite meaning.

Exemplary mTOR inhibitors include, but are not necessarily limited to a class of drugs known as rapalogs. Rapalogs include, but are not necessarily limited to rapamycin and its analogs, such as sirolimus, temsirolimus, everolimus, and ridaforolimus. In specific embodiments, the preferred rapalog is rapamycin.

In other embodiments, mTOR inhibitors may comprise Torin 1 and Torin 2. GABA-T (GABA transaminase) is an enzyme that metabolizes and degrades GABA. A GABA-T inhibitor is an enzyme inhibitor that acts upon GABA-T, inhibiting its function. Examples of GABA-T inhibitors include, but are not necessarily limited to, valproic acid, vigabatrin, phenylethylidenehydrazine, ethanolamine-O-sulfate (EOS), and L-cycloserine. In specific embodiments, the preferred GABA-T inhibitor is vigabatrin.

In specific embodiments, combination therapies may comprise the administration of therapeutically effective amounts of a composition comprising a gene encoding a functional SSADH enzyme operably linked to a targeting vector, Torin 2, and Vigabatrin.

In some embodiments, mTOR inhibitor and/or GABA-T inhibitor may be administered in doses ranging from 1-25 μg per kg of body weight per day. In some embodiments, the mTOR inhibitor and/or GABA-T inhibitor may be administered two times a day. In some embodiments, the mTOR inhibitor and/or GABA-T inhibitor may be administered three times a day.

In alternative embodiments, the invention comprises methods of treating SSADHD in a subject in need thereof, comprising administering a therapeutically effective amount of Na—K—Cl cotransporter 1 (NKCC1) inhibitor to the subject.

NKCC proteins are membrane transport proteins that transport sodium, potassium, and chloride ions across the cell membrane. Because they move each solute in the same direction, NKCC proteins are considered symporters. They maintain electroneutrality by moving two positively charged solutes (sodium and potassium) alongside two parts of a negatively charged solute (chloride). NKCC1 is widely distributed throughout the human body, especially in organs that secrete fluids, called exocrine glands.

NKCC1 is also expressed in many regions of the brain during early development, but not in adulthood. This change in NKCC1 presence seems to be responsible for altering responses to the neurotransmitters GABA and glycine from excitatory to inhibitory, which was suggested to be important for early neuronal development. As long as NKCC1 transporters are predominantly active, internal chloride concentrations in neurons is raised in comparison with mature chloride concentrations, which is important for GABA and glycine responses, as respective ligand-gated anion channels are permeable to chloride. With higher internal chloride concentrations, outward driving force for this ions increases, and thus channel opening leads to chloride leaving the cell, thereby depolarizing it. Later in development expression of NKCC1 is reduced, while expression of a KCC2 K—Cl cotransporter increased, thus bringing internal chloride concentration in neurons down to adult values.

As described in Example 3, postnatal seizures manifested in SSADHD patients may be caused by overexpression of NKCC1. Accordingly, inhibition of NKCC1 might have positive therapeutic effects in SSADHD. In some embodiments, SSADHD may be treated by administering a therapeutically effective amount of an NKCC1 inhibitor to a subject in need thereof.

Suitable NKCC1 inhibitors include, but are not necessarily limited to, bumetanide, allopregnanolone, pregnanolone, progesterone, gaboxadol, etifoxine, XBD-173, FG-7142, gabazine, isoniazid, encenicline, and AVL-3288. In specific embodiments, the NKCC1 inhibitor is bumetanide.

The invention is further described in the following examples, which do not limit the scope of the invention described in the claims.

### EXAMPLES

### Example 1—NCS-382, a Potential Novel Therapeutic for SSADHD

Pharmacological and Structural Considerations

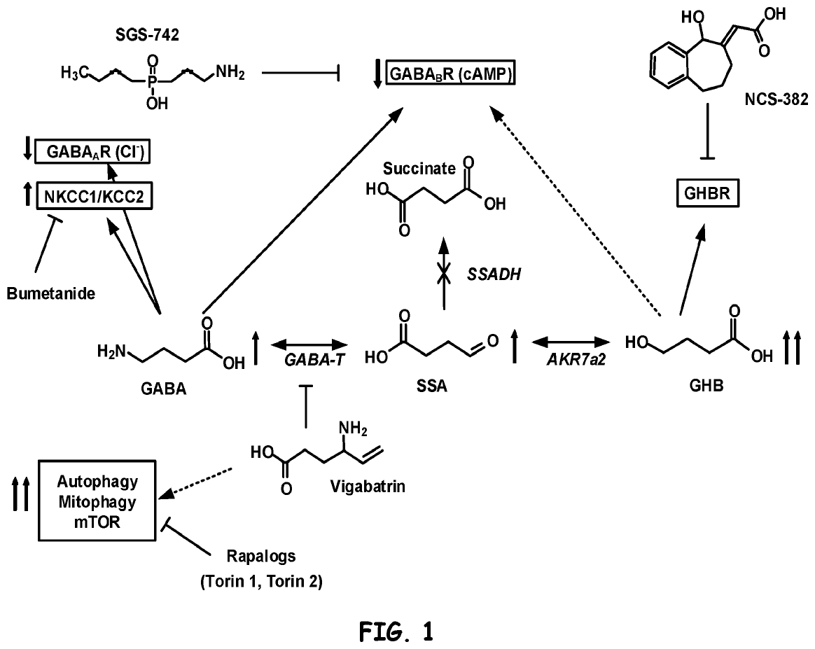

NCS-382 is a putative GHB receptor (GHBR) antagonist with a Ki 14 times lower than that of GHB (FIG. 1) (Maitre Prog Neurobiol 51:337-361 (1997); Vogensen et al. J Med Chem 56:8201-8205 (2013)), and may be the only known antagonist of GHBRs (Bay et al. Biochem Pharmacol 87:220-228 (2014)), whose molecular structure(s) remain undefined. NCS-382 was effective in rescuing aldh5a1−/− mice from premature lethality and in blocking the motor deficits induced by the GHB prodrug, gamma-butyrolactone (GBL) (Ainslie et al. Pharmacol Res Perspect 4:e00265 (2016); Gupta et al. J Pharmacol Exp Ther 302:180-187 (2002)). NCS-382 exists as a racemic mixture (hydroxyl-carbon; FIG. 1). The R-isomer is twice as potent as the racemic mixture, and 13-fold more potent than the S-enantiomer (Castelli et a1. CNS Drug Rev 10:243-260 (2004)). Prior to Applicants' initial studies described below, preclinical pharmacokinetic/toxicological analyses of NCS-382, mandatory considerations prior to clinical intervention, had not been reported.

Pharmacokinetic and Toxicology of NCS-382, and Potential

Limited earlier studies on the pharmacodynamic characteristics of NCS-382 were performed in baboon and pigeon, and were designed to exploit antagonist specificity for the GHBR in order to interrogate the central effects of GHB (Quang et al. Life Sci 71:771-778 (2002); Castelli et al. J Neurochem 87:722-732 (2003); Castelli et al. CNS Drug Rev 10:243-260 (2004)). Applicants obtained detailed pharmacokinetic measures following i.p. administration of NCS-382 (100 to 500 mg/kg) in C57/B6 mice (Ainslie et al. Pharmacol Res Perspect 4:e00265 (2016)). The plasma elimination t½ for NCS-382 ranged from 0.25-0.68 h in a dose-dependent fashion. Brain residence for NCS-382 was longer, with t½ ranging from 0.76-0.97 h, and decreasing with increasing dose. The brain-to-plasma ratio based on area under the concentration-time curves (AUCs) ranged from 0.72-1.8. The trend for decreased brain t½ with increasing dose may reflect saturation of central GHB binding sites, leaving more unbound NCS-382 for return to the systemic circulation. The fraction of total NCS-382 dose recovered in the urine was low (<4%), and undetectable in the feces (<150 ng/mg feces), suggesting metabolism as the primary route of elimination featuring glucuronidation (major product) and dehydrogenation (minor product) at the hydroxyl-moiety (FIG. 1). The intrinsic clearance of NCS-382 (Clint) in the presence of NADPH and assessed by monitoring parent disappearance was 0.587 and 0.513 mL/min/mg protein in mouse and human liver microsomes (MLMs, HLMs), respectively. Calculated murine and human hepatic clearances were 5.2 and 1.2 L/h/kg body weight, respectively. The Michaelis constant (Km) for dehydrogenation was 29.5±10 and 12.7±4.9 μM in mouse and human, respectively. Glucuronide formation was linear in both species up to 100 μM. UGT2B7 (uridine 5′-diphosphoglucuronosyltransferase; UDP-glucuronosyltransferase 2B7) was suspected as the primary isoform of glucuronidating enzymes responsible for NCS-382 metabolism, based upon competition studies with the UGT2B7 inhibitor, diclofenac (Ainslie et al. Pharmacol Res Perspect 4:e00265 (2016)). Co-administration of diclofenac (25 mg/kg) improved the efficacy of NCS-382 (300 mg/kg) to block the sedative and motor effects in animals treated with GBL. Plasma levels of glucuronides of NCS-382 and diclofenac decreased in combinatorial treatment relative to mice receiving either agent alone.

The identity of drug metabolizing enzymes active in the biotransformation(s) of NCS-382 also has not been reported, nor has the ability of NCS-382 to inhibit the typical enzymes, namely cytochrome P450s (CYP), involved in the metabolism of drugs. Accordingly, Applicants employed HLMs and FDA-recommended probe substrates in reactions catalyzed by seven CYP isoforms (CYP I A2, 2B6, 2C8, 2C9, 2C19, 2D6 and 3A4) (Vogel et al. Toxicol In Vitro 40:196-202 (2017)). NCS-382 did not inhibit any of the tested enzymes at the highest tested dose (30 μM). Further, NCS-382 manifested minimal capacity to induce nuclear receptors involved in drug biotransformation and transport (aryl hydrocarbon, constitutive androstane, and pregnane X receptors) at supraphysiological doses (up to 500 μM). Collectively, these findings indicate a low-risk for CYP P450-mediated drug-drug interactions.

HepG2 cells were subsequently used to examine the cellular toxicity of NCS-382 at up to 1 mM. Multiple biomarkers assessing cellular integrity, survival, and organelle function revealed little evidence for NCS-382 cytotoxicity (Vogel et al. Toxicol In Vitro 40:196-202 (2017)). Gene expression studies using NCS-382 in HepG2 cells revealed only a minor number of genes (of 370 tested) showing dysregulation (Table 1). Additionally, high-dose NCS-382 demonstrated only minimal pharmacotoxicity in neural stem cells (NSCs, or neuronal progenitor cells) derived from aldh5a1−/− mice (e.g., aldh5aJ−/− NSCs) (Vogel et al. Toxicol In Vitro 46:203-212 (2017)). These cells were developed as an in vitro model of SSADHD, showing increased GHB content in culture medium, enhanced biomarkers of oxidative stress and increased mitochondrial number and highlighting the utility of NSCs as a useful preclinical screening tool for evaluating therapeutics for SSADHD (Vogel et al. PLoS One 12(10):e0186919 (2017)). In sum, although a number of additional studies will be needed, pilot pharmacokinetic/safety/toxicological evaluations support the potential for clinical application of NCS-382 in SSADHD.

TABLE 1
Genes altered >4-fold by NCS-382 (0.5 mM) in HepG2 cells.

| Gene | Gene Product | Function | Gene Grouping | Change |
|---|---|---|---|---|
| CD36 | CD36 Molecule | Involved in platelet | Steatosis | ↓ |
| | | activation, signaling | | |
| | | and aggregation and | | |
| | | metabolism | | |
| HTRA4 | High-Temperature | Degrades misfolded | ER Stress & | ↑ |
| | Requirement | secretory proteins | unfolded protein | |
| | Factor A4 | | response | |
| SERPINA3 | Serpin Family A | plasma protease | Phospholipidosis | ↓ |
| | Member 3 | inhibitor; deficiency | | |
| | | has been associated | | |
| | | with liver disease | | |
| SLC2A3 | Glucose | Facilitative glucose | Phospholipidosis | ↓ |
| | Transporter | transporter | | |
| | Type 3, Brain | | | |
| SLC51A | OST-Alpha | Transports the major | Cholestasis | ↓ |
| | | species of bile acids | | |
| TNFRSF1A | Tumor Necrosis | Activates NF-kB, | Apoptosis & | ↑ |
| | Factor Receptor | mediates apoptosis, | Necrosis | |
| | Superfamily, | and functions as a | | |
| | Member 1A | regulator of | | |
| | | inflammation | | |

Preclinical Efficacy of NCS-382 in aldh5a1−/− Mice

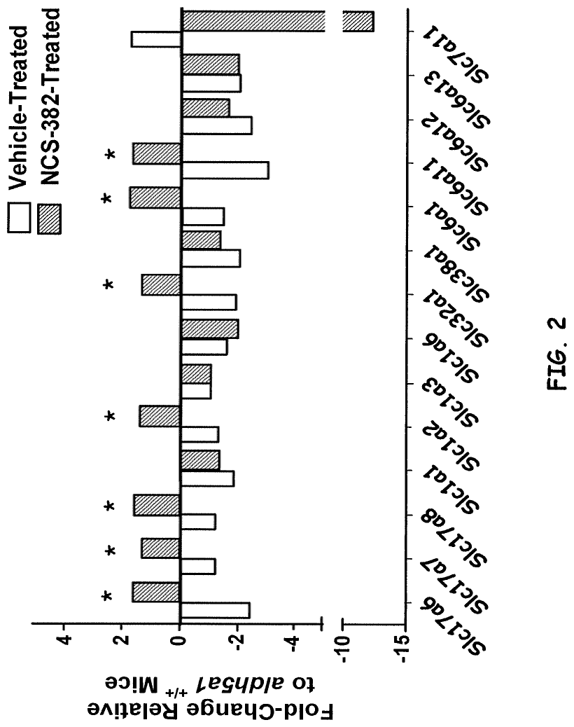

Applicants subsequently turned attention to the transport of NCS-382 in vitro. The objective here was to investigate the potential of NCS-382 to block uptake of GHB. Applicants demonstrated for the first time using Madin-Darby Canine Kidney (MDCK) cells that NCS-382 is actively transported and capable of inhibiting GHB transport (Vogel et al. Toxicol In Vitro 46:203-212 (2017)). Following these in vitro assays with in vivo studies in aldh5a1−/− mice, Applicants found the ratio of brain/liver GHB to be unaffected by chronic NCS-382 administration (300 mg/kg; 7 consecutive days), which appeared paradoxical. This finding suggests that potential future application of NCS-382 may only be modestly beneficial since brain GHB levels do not appear to be modified with chronic treatment. Applicants examined cortical regions from the NCS-treated mice and evaluated the expression of a number of solute carriers involved in neurotransmitter transport. As shown in FIG. 2., Applicants found essentially all of these transporters down-regulated in aldh5a1−/− cortex in the absence of treatment. NCS-382 normalized the aberrant expression of seven of these carriers, including both glutamate and GABA transporters, had no effect on six and actually induced significant down-regulation of the glutamate-cystine antiporter. This finding is of interest in view of the significant depletion of glutathione in this animal model, the observation that glutathione is composed of glutamate, cysteine and glycine, and the earlier finding that glutamate/glutamine levels are abnormal in aldh5a1−/− brain (Gupta, J Pharmacol Exp Ther 302:180-187 (2004); Chowdhury, (2007)). These results provide modest preclinical support for the use of NCS-382 in SSADHD. Additional in vivo studies are in progress in aldh5a1−/− mice using NCS-382, assessing lifespan, body weight and neurobehavioral outcomes, and using both chronic and acute administration paradigms.

### Example 2—Enzyme Replacement Therapy for SSADHD

As a therapeutic approach, enzyme replacement therapy (ERT) has gained prominence in the lysosomal storage disorders, although it should be feasible in organic acidernias (Darvish-Damavandi et al. Mol Genet Metab Rep 8:51-60 (2016)). Applicants examined the feasibility of ERT in aldh5a1−/− mice using a GST (glutathione)-tagged human ALDH5A1 gene construct that overexpresses the GST-hSSADH fusion protein with accompanying ampicillin resistance in E. coli (DNASU Plasmid Repository; Ramachandran et al. (2004)). Crude extracts of E. coli were harvested that had been transfected and grown in standard LB broth supplemented with ampicillin at 37° C. overnight, pelleted and lysed with Pefabloc (protease inhibitor; Sigma-Aldrich, St. Louis, Mo. USA) and lysozyme. GST-hSSADH resident in crude lysates of E. coli was purified using Pierce™ GST spin purification kits, followed by dialysis and subsequent concentration using polyethersulfone (PES) columns. Resultant protein content was quantified using a standard BCA protein assay. The GST-tagged enzyme activity was cleaved with thrombin and the activity of ALDH5A1 determined employing spectrofluorometry based on the NAD/NADH couple (Gibson et al. Clin Chim Acta 196:219-221 (1991)).

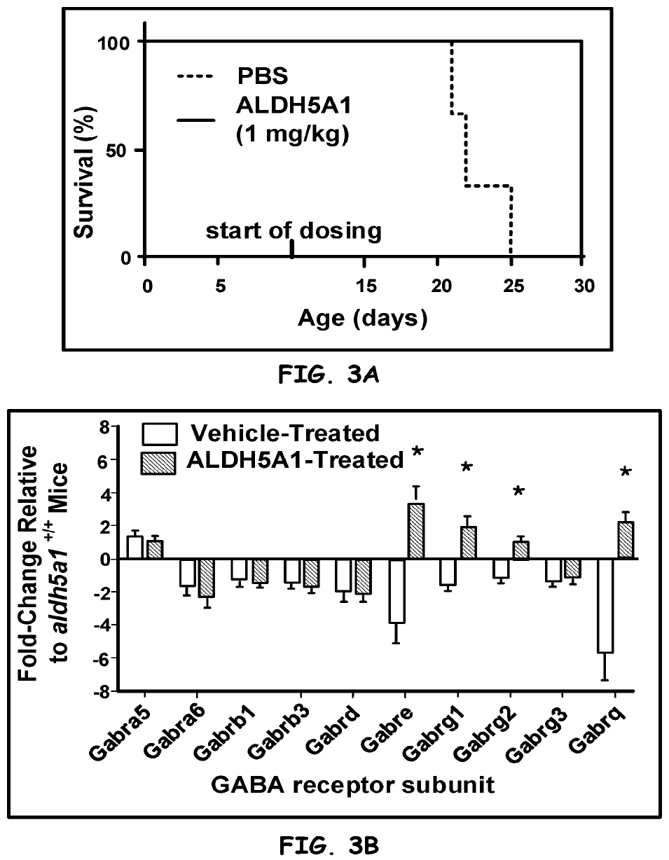

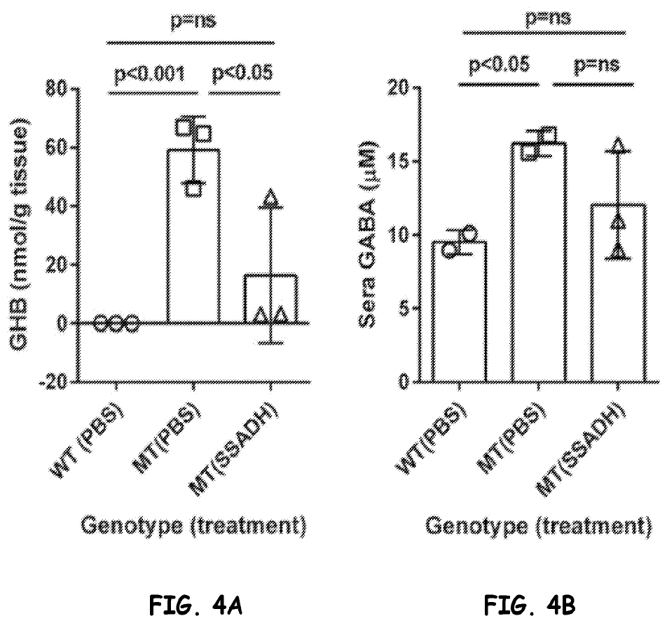

The feasibility of ERT using bacterially-produced ALDH5A1 was subsequently evaluated in aldh5a1−/− mice. As endpoint, Applicants employed rescue of this model from premature lethality (day of life (DOL) 21-23; endpoint, DOL 30, which is highly significant survival), the latter endpoint chosen in view of the limited availability of treatment protein. Purified ALDH5A1 was administered (i.p., 1 mg/kg in PBS, q.d.) beginning at day of life 10. Median survival of vehicle-treated aldh5a1−/− mice was 22 days compared to 80% survival rate (4 of 5 mice) to 30 days with ERT treatment (Logrank; p 0.04; FIG. 3 (inset)). Brain, liver and sera were collected from surviving ERT-treated subjects harvested at DOL 30. The expression of GABA receptor genes was contrasted between ERT (DOL 30) and untreated (DOL 21) aldh5a1−/− mice, with data normalization to DOL 21 aldh5a1+/+ mice. The expression of several GABAA receptor subunits (primarily gamma, epsilon and theta) were significantly corrected in aldh5a1−/− mice with enzyme intervention (FIG. 3). Since Applicants hypothesized that parenterally administered SSADH would lower metabolites (GHB, GABA) in blood and other tissues, these intermediates were quantified (FIG. 4) in sham and enzyme-treated subjects using LC/MS-MS (Gibson et al. Biomed Environ Mass Spectrom 19:89-93 (1990); Kok et al. J Inherit Metab Dis 16:508-512 (1993)). Although the numbers were low, Applicants found a significant correction of GHB in the brain of enzyme treated animals, and a trend toward improved levels of GABA in blood. This promising trial was not sufficiently powered for biochemical measures, and more extensive evaluations are needed with larger n values. In particular, Applicants will assess the levels of SSADH activity in blood and organs, and, PEGylation may be utilized to increase protein t½ and reduce immunogenicity (Bell et al. PLOS ONE 12:e0173269 (2017)).

### Example 3—the Paradox of Seizures in a SSADHD, a HyperGABAergic Disorder

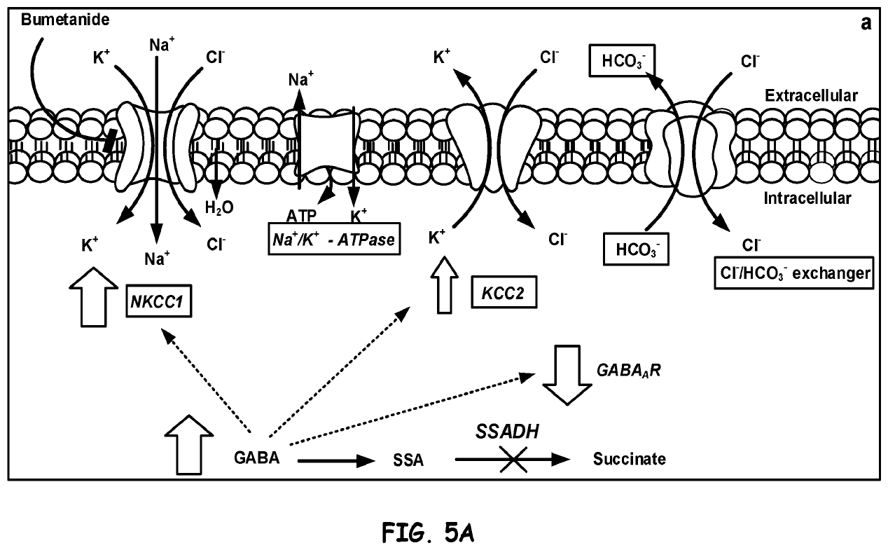

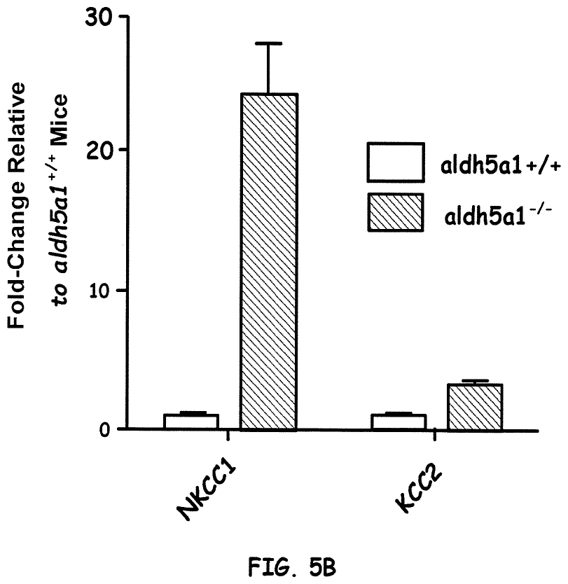

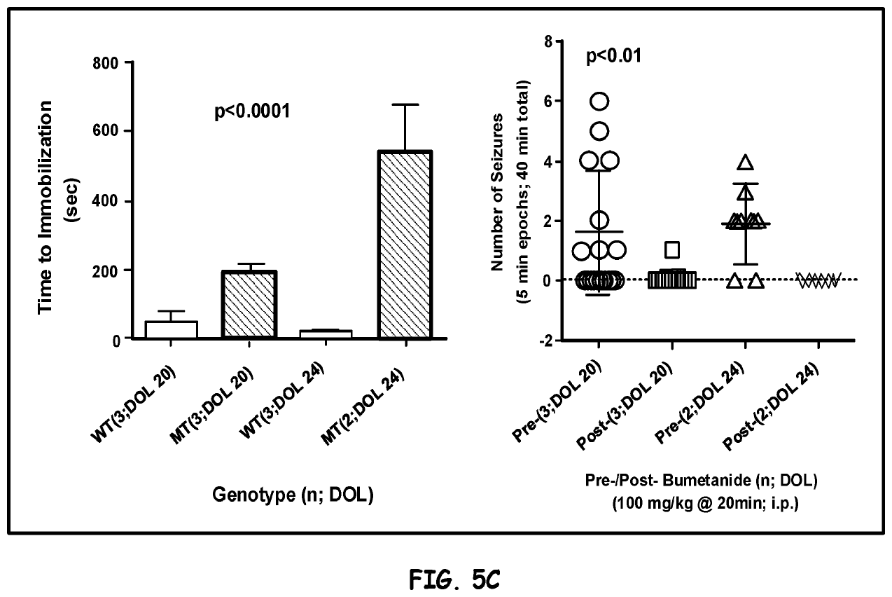

Chloride directional flux through the GABAA receptor in mammalian brain is regulated by the transmembrane chloride gradient which is itself controlled primarily by two transporters, the sodium-potassium-chloride symporter (NKCC1) and the potassium-chloride cotransporter (KCC2) (FIG. 5A) (Kilb Neuroscientist 18:613-630 (2012)). Activation of the GABAA receptor when the activity of NKCC1 is increased and intracellular chloride concentrations are elevated results in chloride efflux, plasma membrane depolarization and paradoxical neurotransmission activation. Such a situation is observed in the fetal brain but is reversed postnatally when GABAA receptor activation consistently leads to increased chloride cellular uptake, hyperpolarization and neurotransmission inhibition. Applicants hypothesized that in SSADHD, postnatal seizures were caused by overexpression of NKCC1 and a continuing excitatory capacity of GABAA receptors after birth. If confirmed, this mechanism would explain the paradox of seizures in a hyperGABAergic condition (Vogel et al. Pediatr Neurol 66:44-52.e1. (2017)). Furthermore, it would also suggest that inhibition of NKCC1 might have positive therapeutic effects in SSADHD. In support of these hypotheses, Applicants found that NKCC1 was highly overexpressed in aldh5a1−/− brain (FIG. 5B). The expression of KCC2, which transports chloride ions out of the cell, was also increased but significantly less than that of NKCC1 (FIG. 5B). Hence, the NKCC1 to KCC2 ratio was significantly increased, suggesting that intracellular chloride concentrations might be increased in the aldh5a1−/− brain, favoring a depolarizing and excitatory activity as the predominant role for GABAA receptors. This remains to be quantitatively evaluated employing neurophysiological assessments in vitro via patch-clamp methodology, or perhaps in vivo using two-photon measurement with chloride ion probes.

The potential therapeutic role of NKCC1 inhibition in SSADHD was next examined. In this context, Applicants postulated that there would be resistance to the sedative activity of NKCC1 inhibitors in SSADHD, because of the increased expression of NKCC1. Applicants treated aldh5a1+/+ and aldh5a1−/− mice with acute i.p. doses of bumetanide (25 and 100 mg/kg body weight) with video recording of seizure activity as well as assessment of the time to immobilization. Bumetanide is a known inhibitor of both NKCC1 and 2 originally approved for edema but with demonstrated antiepileptic efficacy in several neurological/epileptic disorders despite poor brain penetration (Levy et al. Curr Emerg Hosp Med Rep 1(2) doi:10.1007/s40138-013-0012-8. (2013); Oliveros et al. Pediatr Crit Care Med 12:210-214 (2011); Rahmanzadeh et al. Schizophr Res 184:145-146 (2016); Cleary et al. PLOS ONE 8:e57148 (2013)). Applicants employed Pinnacle technology (https://www.pinnaclet.com) technology for recording of animal behavior in conjunction with a rubric developed by Noldus technology to evaluate seizure activity (http://www.noldus.com/animal-behavior-research). Applicants' design was to quantify generalized tonic-clonic seizures in 5 minute epochs for 40 minutes in continuum. At the 20 minute mark, a single intraperitoneal administration of bumetanide (25 or 100 mg/kg) was given and the animals returned to the open field setting. A dose of 25 mg/kg bumetanide did not induce immobilization during the 40 minute recording period for either aldh5a1+/+ or aldh5a1−/− mice. Conversely, 100 mg/kg resulted in rapid induction of immobilization (FIG. 5C). Of interest, however, was the observation that aldh5a1−/− were significantly more resistant to the effect of bumetanide, at both DOL 20 and 24, with this resistance about 3-fold greater in the older aldh5a1−/− mice. In addition, no seizure activity was observed in the mutant mice in the time period between bumetanide injection and the onset of immobilization, an approximate period of 3-8 minutes. As well, the lower dose of bumetanide (25 mg/kg) also reduced seizure activity in aldh5a1−/− mice without sedation (data not shown). These preliminary data suggest the resistance of the aldh5a1−/− mice to the sedative effect of bumetanide is secondary to increased NKCC1 activity and disruption in the chloride gradient, suggesting that manipulation of NKCC1 and restoration of intracellular chloride homeostasis represents a potentially attractive therapeutic target in SSADHD.

### Example 4—GABA and mTOR: Treatment Strategies and Pathophysiological Insights in SSADHD

Inhibition of mTOR, a Therapeutic Target in SSADHD

Increased mitochondrial numbers (including both the size and total number of organelles) were first documented in aldh5a1−/− mice in pyramidal hippocampal neurons (Nylen et al. 2009). Subsequently, Lakhani and coworkers (EMBO Mol Med 6:551-566 (2014)) demonstrated that increased GABA levels in S. cerevisiae led to activation of mTOR (molecular target of rapamycin), manifesting as elevated mitochondrial numbers and enhanced oxidative stress. This same group of investigators further documented that mitochondrial numbers in brain and liver derived from aldh5a1−/− mice were increased and associated with enhanced oxidant stress, all of which could be normalized by the mTOR inhibitor, rapamycin.

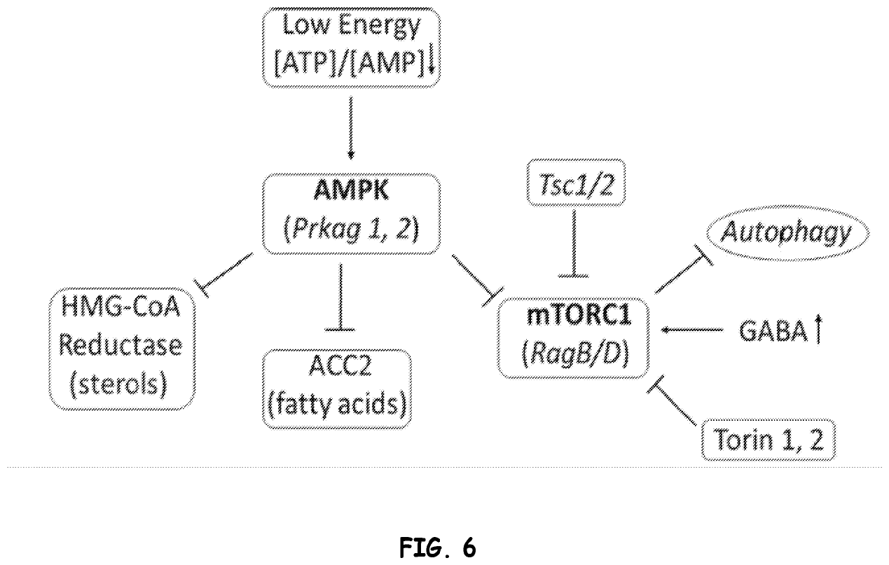

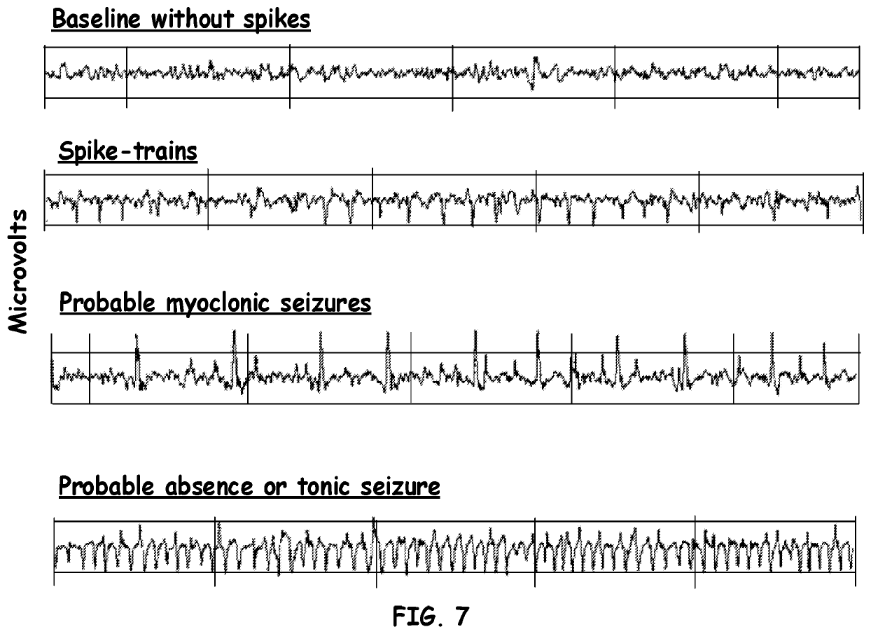

These studies were the genesis of further preclinical interventional studies in aldh5a1−/− mice with mTOR inhibitors (rapamycin, temsirolimus), dual mTORC1/2 and PI3K inhibitors, as well as mTOR-independent autophagy inducing drugs (Vogel et al. J Inherit Metab Dis 39:877-886 (2016); FIG. 6). Lifespan extension (rescue from premature lethality) for aldh5a1−/− mice was observed across a number of mTOR-specific and dual inhibitors, and findings with the dual inhibitor XL765 and Torin2 were striking. XL765 induced a modest weight improvement over 35 days until sacrifice at DOL 50 in aldh5a1−/− mice (Vogel et al. J Inherit Metab Dis 39:877-886 (2016)). Conversely, mTOR-independent inducers of autophagy were without benefit in mitigating premature lethality. These data suggest that induction of autophagy through mTOR-independent mechanisms was insufficient for rescue, suggesting that other functions associated with mTOR may be involved in the clinical efficacy of mTOR blockade in aldh5a1−/− mice.

Studies with Torin 2 administration in aldh5a1−/− mice, beginning at DOL10 under an escalating dose paradigm, were evaluated for seizures using a three channel recording electrocorticography (Pinnacle Technology; https://www.pinnaclet.com/eeg-emg-systerns.html) and electrode-implanted aldh5a1+/+ and aldh5a1−/− mice. EEG was scored offline using semi-automated detection (Neuroscore, Data Sciences International, St. Paul, Minn.) for seizure frequency (total number of seizures), and total ictal time (cumulative time spent in seizures); where a seizure event was defined as a run of continuous spikes ≥3s in duration on the EEG. Automatically detected events were verified by visual inspection (Dhamne et al. Mol Autism 8:26 (2017)). Our prediction was that Torin 2 administration would lead to improvement in both parameters. Applicants found that Torin 2 was ineffective at reducing seizure frequency in aldh5a1−/− mice (Table 2), and unexpectedly found that it significantly extended the total ictal time (Table 2). It is of interest that Torin 2 corrected (upregulated) a number of GABA(A)ergic receptor subunits (Vogel et al. J Inherit Metab Dis 39:877-886 (2016)). If these receptors remain immature, consistent with our hypothesis for bumetanide (see above), then their subsequent up-regulation could conceivably exacerbate depolarization, and thus enhance cumulative epileptic outburst duration, as Applicants observed (Table 2). On the other hand, others have provided evidence that mTOR inhibitors prevent the sprouting of mossy fibers, a form of synaptic reorganization which occurs in epilepsy and can lead to the formation of recurring excitatory circuits (Dudek et al. 2017). Which mechanism explains enhanced seizure activity with Torin 2 remains under investigation.

TABLE 2
Effect of Torin 2 on Seizure Frequency and
Total Ictal Time in aldh5a1^{+/+} and aldh5a1^{-/-} Mice.

| | | |
|---|---|---|
| | Torin 2 | t test |
| Vehicle | (10 mg/kg) | (Veh. vs. Torin) |

Seizure Frequency (avg/hr)

| | | | |
|---|---|---|---|
| aldh5a1^{+/+} | 5.4 ± 0.8 | 0.50 ± 0.59 | p = 0.12 |
| aldh5a1^{-/-} | 3.2 ± 0.8 | 9.6 ± 1.3 | p = 0.11 |
| t test | p = 0.41 | p = 0.23 | |

Total Ictal Time (seconds) (avg/hr)

| | | | |
|---|---|---|---|
| aldh5a1^{+/+} | 22 ± 1.7 | 2.1 ± 1.2 | p = 0.15 |
| aldh5a1^{-/-} | 19 ± 2.0 | 282 ± 8.0 | p = 0.03 |
| t test | p = 0.90 | p = 0.30 | |

Data depicted as mean ± SEM. Seizure events included spike-trains, probably myoclonic as well as absence and tonic-clonic seizures.

### Example 4—Future Directions

Dissecting the mTOR Role Using Vigabatrin-Treated and aldh5a1^{-/-} Mice.

Vigabatrin (VGB)-treated mice represent a drug-induced form of GABA-transaminase deficiency, since VGB irreversibly inhibits this first enzyme of GABA metabolism (FIG. 1). Employing VGB, significantly elevated GABA in the CNS can be achieved at relatively low daily doses (˜10 mg/kg), or via chronic subcutaneous delivery with calibrated osmotic minipumps (Vogel et al. J Inherit Metab Dis 39:877-886 (2016); Vogel et a1. Toxicol In Vitro 40:196-202 (2017)). A cardinal difference between these “models” resides in the absence of elevated GHB in VGB-treated mice, which enables us to begin to more clearly isolate the role of GABA and its effect on mTOR. Applicants have begun exploring this aspect employing gene expression in these different models.

mTOR coordinates numerous intracellular bioenergetic cues that serve to control growth and catabolism (i.e. translation, autophagy) (FIG. 6). A number of gene expression changes correlated between the two animal models. Prkag1 was down-regulated in brain of both models (Vogel et al. Toxicol In Vitro 40:196-202 (2017); Vogel et al. Pediatr Neurol 66:44-52.e1. (2017)). Prkag1 is a gamma regulatory subunit of the heterotrimeric AMP-activated protein kinase (AMPK), which also contains an alpha catalytic subunit and a non-catalytic beta subunit (FIG. 6). AMPK is an important energy-sensing enzyme that monitors cellular energy status. In response to cellular metabolic stresses, AMPK is activated, and thus phosphorylates and inactivates acetyl-CoA carboxylase (ACC) and 3-hydroxy 3-methylglutaryl-CoA reductase (HMGCR), key enzymes involved in regulating de novo biosynthesis of fatty acid and cholesterol. AMPK acts via direct phosphorylation of metabolic enzymes; and acts longer-term via phosphorylation of transcription regulators. During low energy conditions AMPK is a critical negative effector of mTOR activation.

Prkag2 was also down-regulated in aldh5a1−/− brain (and downregulated in VGB-treated mouse eye). Decreased expression of Prkag1 and 2 was normalized by the mTOR inhibitor Torin 1, and correction of AMPK's activator (downregulated in aldh5a1−/− mice), Stk11, was normalized by Torin2. As well, Tsc1 and 2, signaling systems downstream of AMPK, also exhibited lower expression which was normalized by Torin 1 and Torin 2 in aldh5a1−/− mouse brain. Rag GTPases activate mTOR through its amino acid sensing pathway, and RagB/RagD expression was upregulated in aldh5a1−/− brain (RagB was upregulated in VGB-treated eye tissue, while RagD was upregulated in VGB-treated brain tissue). Together, these findings strongly suggest that correction of AMPK is involved in the pro-survival effects of Torin drugs in aldh5a1−/− mice. Understanding the potential clinical utility of mTOR inhibitors to treat disorders of GABA metabolism will continue to be a central theme in our laboratory.

Given the multisystem dysfunction in SSADHD, it is likely that combinatorial therapies will be required to leverage incremental improvements in the phenotype. A logical starting point would be VGB, if the issue of ocular toxicity could be overcome. Indeed, with regard to this, inhibitors of mTOR might be added to mitigate the effects of additionally increased GABA associated with VGB. An antioxidant agent would also be of value, given the evidence for oxidative stress in this disorder (Gupta et al. J Pharmacol Exp Ther 302:180-187 (2002)). As shown in the current report, ERT may have therapeutic benefit, which could be combined with genetic manipulations in the future, such as CR1SPRCas9 approaches. Nonetheless, Applicants' short-term goals remain the development of targeted therapy for SSADHD.

Various modifications and variations of the described methods, pharmaceutical compositions, and kits of the invention will be apparent to those skilled in the art without departing from the scope and spirit of the invention. Although the invention has been described in connection with specific embodiments, it will be understood that it is capable of further modifications and that the invention as claimed should not be unduly limited to such specific embodiments. Indeed, various modifications of the described modes for carrying out the invention that are obvious to those skilled in the art are intended to be within the scope of the invention. This application is intended to cover any variations, uses, or adaptations of the invention following, in general, the principles of the invention and including such departures from the present disclosure come within known customary practice within the art to which the invention pertains and may be applied to the essential features herein before set forth.

1. A method of treating succinic semialdehyde dehydrogenase deficiency (SSADHD) in a subject in need thereof, comprising administering to the subject a composition comprising an enzyme suitable for enzyme replacement therapy, wherein the enzyme suitable for enzyme replacement therapy is a succinic semialdehyde dehydrogenase (SSADH) enzyme.

2. The method of claim 1, wherein the succinic semialdehyde dehydrogenase enzyme is a human Aldehyde Dehydrogenase 5 Family Member A1 (ALDH5A1).

3. The method of claim 1, wherein the enzyme is administered at a range of 1-10,000 micrograms functional SSADH enzyme per kg of body weight per day.

4. The method of claim 1, wherein the composition is administered once per week, bi-weekly, or once a month.

5. The method of claim 1, wherein the composition is administered intravenously.

6. The method of claim 1, wherein the composition is administered intrathecally.

7. The method of claim 1, wherein the SSADH enzyme is PEGylated.

8. The method of claim 1, further comprising administering one or more of an mTOR inhibitor, a GABA-T inhibitor, or a combination thereof to the subject.

9. The method of claim 8, wherein the mTOR inhibitor comprises one or more of rapamycin, sirolimus, temsirolimus, everolimus, ridaforolimus, Torin 1, and Torin 2.

10. The method of claim 8, wherein the mTOR inhibitor is rapamycin.

11. The method of claim 8, wherein the GABA-T inhibitor is vigabatrin.

12. The method of claim 8, comprising administering a therapeutically effective amount of at least one of Torin 2 or Vigabatrin.

13. The method of claim 12, wherein the therapeutically effective amount of at least one of Torin 2 or Vigabatrin comprises 1-25 micrograms per kg of body weight per day.

14. The method of claim 12, wherein at least one of Torin 2 or Vigabatrin are administered two or three times a day.

15. The method of claim 1, wherein the subject has increased levels of circulating metabolites.

16. The method of claim 15, wherein the circulating metabolites are GHB, GABA, or both.
